(19) United States
(12) Patent Application Publication
(10) Pub. No.: US 2016/0000833 A1
(43) Pub. Date: Jan. 7, 2016

(54) METHOD OF PREPARING DEER BONE EXTRACT HAVING INCREASED AMOUNT OF GANGLIOSIDE

(71) Applicant: NONG SHIM CO., LTD., Seoul (KR)

(72) Inventors: Jong Hoon Kim, Goyang-si (KR); Ho Bong Lee, Seoul (KR); Seo Jin Kim, Seoul (KR); Sang Cheol Oh, Seoul (KR); Soo Hyun Park, Seongnam-si (KR); Sung Ug Jung, Seoul (KR); Byung Jung Min, Seoul (KR); Byoung Oon Jeon, Suwon-si (KR); Chang Won Ahn, Bupyeong-gu (KR)

(21) Appl. No.: 13/769,085

(22) Filed: Feb. 15, 2013

(30) Foreign Application Priority Data
Nov. 1, 2012 (KR) 10-2012-0122871
Jan. 9, 2013 (KR) 10-2013-0002610

(51) Int. Cl. A61K 35/32

(52) U.S. Cl. CPC A61K 35/32

(57) ABSTRACT

Disclosed herein is a method of preparing a deer bone extract in which deer bones are subjected to extraction at normal pressure and extraction under pressure to minimize decomposition of deer bone-derived gangliosides by heat and to increase protein extraction yield.

### CROSS-REFERENCE TO RELATED APPLICATION(S)

This application claims the benefit of Korean Patent Application No. 10-2012-122871, filed on Nov. 1, 2012 and Korean Patent Application No. 10-2013-2610, filed on Jan. 9, 2013 in the Korean Intellectual Property Office, the disclosure of which is incorporated herein by reference.

### BACKGROUND

1. Field

Embodiments of the present invention relate to a method of preparing a deer bone extract in which deer bones are subjected to extraction at normal pressure and extraction under pressure in order to minimize decomposition of deer bone-derived gangliosides by heat and to increase protein extraction yield.

2. Description of the Related Art

According to Shennong classics of material medic, deer bones have a warm temper and are sweet-tasting and non-toxic, and thus, when administered after boiling, the boiled deer bones are capable of treating all kinds of diseases caused by muscle weakness and poor bone function due to general weakness in the body.

Recently, it has been discovered that lean meat and general bones of deer have also contain functional ingredients. In particular, analysis results show that deer bones contain approximately 40% gangliosides, which are known to be contained only in deer antlers. As a result of analysis of ganglioside ingredients, it has been confirmed that deer bones contain 5.66 mg/g of gangliosides, male deer bones contain 0.77 to 2.87 mg/g of gangliosides, and elk deer bones contain 0.92 to 2.31 mg/g thereof. Gangliosides are known to maintain and activate physical function, which activates brain cells together with hemoglobin to thus enhance brain function, improve memory and concentration, and boost immunity. In addition, recent clinical data report that gangliosides improve Parkinson's symptoms and relieve diabetic peripheral neuropathy.

A deer bone extract contains large amounts of main ingredients involved in skeletal formation and metabolism, i.e., protein, phospholipids, and the like and also contains chondroitin, collagen, and the like, and thus, it helps bone and joint health. In addition, the deer bone extract contains inorganic components, i.e., calcium and phosphorus, and thus enhances nervous system function and facilitates metabolism.

As to research on deer bones, there have been disclosed a method of extracting gangliosides using deer meat and deer bone (Method of preparing a concentrate by using deer meat and deer bone and a food prepared using the same, Korean Patent Application No.: 10-2002-0064443), an extraction method used to prepare a deer bone extract from which fishy odor is removed by including an herbal medicine (Method of preparing a deer bone extract containing an herbal extract, a deer bone extract prepared using the method, and food and medicines including the deer bone extract, Korean Patent Application No.: 10-2004-0030312), an extraction method using deer antlers together (Healthy drink including a deer bone extract and a method of preparing the same, Korean Patent Application No.: 10-2009-0007905), an extraction method using a mixture of deer antlers, deer flesh, and deer bone (Method of preparing a double-boiled deer extract, Korean Patent Application No.: 10-2009-0126427), a method of effectively removing fishy odor of deer bones by extracting protein from a deer bone extract (Method of preparing a deer bone extract, Korean Patent Application No.: 10-2012-0042214), and the like. However, there has been no research into a method of minimizing the decomposition of gangliosides by heat and increasing protein extraction yield by using deer bones alone.

### SUMMARY

Therefore, it is an aspect of the present invention to provide a method of preparing a deer bone extract in which gangliosides, which exhibit poor thermal resistance, are extracted at a maximum level by extraction at normal pressure and are also extracted under pressure to increase protein extraction yield, and thus, the amount of gangliosides and protein extraction yield are increased.

Additional aspects of the invention will be set forth in part in the description which follows and, in part, will be obvious from the description, or may be learned by practice of the invention.

In accordance with one aspect of the present invention, a method of preparing a deer bone extract includes performing hot water extraction on deer bones at normal pressure, performing hot water extraction on the deer bones under pressure, separating oil from the extract, filtering the oil-separated solution, and concentrating the filtrate.

The hot water extraction at normal pressure may be performed at about 95 to about 105° C. for about 3 to about 12 hours after mixing the deer bones and purified water in a weight ratio of about 1:1.5 to about 1:7.

The hot water extraction under pressure may be performed at about 105 to about 140° C. for about 0.5 to about 12 hours after mixing the deer bones and purified water in a weight ratio of about 1:1.5 to about 1:4.

The oil separation may be performed using an oil separator after maintaining the extract at about 60 to about 90° C. for about 0.5 to about 4 hours.

The filtering may be performed using a 60 to 300 mesh filter.

The concentrating may be performed at about 35 to about 70° C. and about 0 to about 160 mmHg.

In accordance with another aspect of the present invention, there is provided a deer bone extract prepared using the method described above.

### BRIEF DESCRIPTION OF THE DRAWINGS

These and/or other aspects of the invention will become apparent and more readily appreciated from the following description of the embodiments, taken in conjunction with the accompanying drawings of which:

FIG. 1 is a flowchart illustrating processes of performing normal pressure extraction, pressurized extraction, oil separation, filtration, concentration, and powdering of deer bones, according to an embodiment of the present invention;

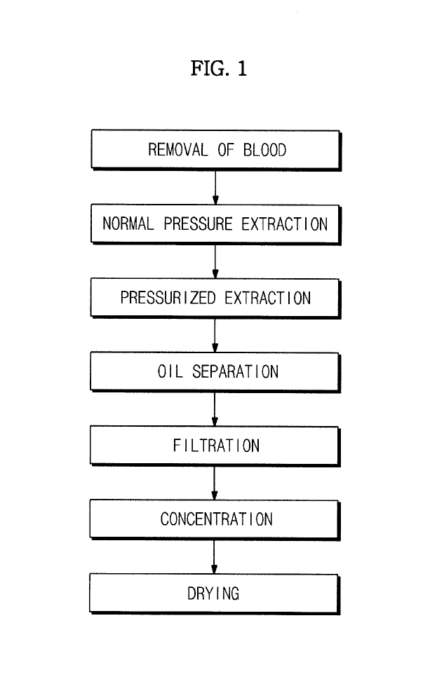

FIG. 2 is a graph showing changes in the total amount of gangliosides according to temperature when deer bones are subjected to extraction, according to an embodiment of the present invention;

FIG. 3 is a graph showing changes in the total amount of gangliosides and solid yield when deer bones are subjected to extraction at normal pressure, according to an embodiment of the present invention;

FIG. 4 is a graph showing a yield of deer bone powder per weight (ton) of an extractor according to the amount of water added when deer bones are subjected to extraction at normal pressure, according to an embodiment of the present invention;

FIG. 5 is a graph showing changes in the total amount of gangliosides according to temperature when deer bones are subjected to normal pressure extraction, according to an embodiment of the present invention;

FIG. 6 is a graph showing changes in the total amount of gangliosides and solid yield when deer bones are subjected to extraction under pressure, according to an embodiment of the preset invention;

FIG. 7 is a graph showing changes in the total amount of gangliosides and solid yield when deer bones are subjected to normal pressure extraction and extraction under pressure, according to an embodiment of the present invention; and.

FIG. 8 is a graph showing a yield of deer bone powder per weight (ton) of an extractor according to the amount of water added when deer bones are subjected to extraction under pressure, according to an embodiment of the present invention.

### DETAILED DESCRIPTION

Reference will now be made in detail to the embodiments of the present invention, examples of which are illustrated in the accompanying drawings, wherein like reference numerals refer to like elements throughout.

A method of preparing a deer bone extract, according to an embodiment of the present invention, will now be described in detail.

First Operation: Removal of Blood from Deer Bones

Deer bones and purified water are mixed in a weight ratio of about 1:1.3 to about 1:3 and blood is removed from the deer bones for about 2 to about 12 hours. This process is repeated two to four times.

Second Operation: Hot Water Extraction at Normal Pressure

The deer bones from which blood has been removed in the first operation and purified water are mixed in a weight ratio of about 1:1.5 to about 1:7 and subjected to extraction at about 95 to about 105° C. for about 3 to about 12 hours. When deer bones are subjected to normal pressure extraction at about 95 to about 105° C. for about 3 to about 12 hours, decomposition of gangliosides by heat may be minimized and the total extraction yield of gangliosides may be increased.

Third Operation: Hot Water Extraction Under Pressure

The deer bones obtained after hot water extraction at normal pressure has been performed in the second operation and purified water are mixed in a weight ratio of about 1:1.5 to about 1:4 and subjected to extraction at about 105 to 140° C. for about 0.5 to about 4 hours. To increase the temperature of an extraction vessel to about 105 to about 140° C., an inner pressure of the extraction vessel is set at about 0.5 to about 3 atm. When the deer bones are subjected to extraction under these conditions, the total amount of gangliosides may be maintained at 500 ppm or more and solids such as protein and the like may be efficiently extracted.

Fourth Operation: Oil Separation

The extracts obtained after the second and third operations are maintained at about 60 to about 90° C. for about 0.5 to about 4 hours and then oil is separated therefrom to remove a lipid layer. When oil separation is performed under these conditions, foaming induced by a large amount of protein is minimized so that oil separation and concentration processes are easily performed.

Fifth Operation: Filtration

The oil-separated solution is filtered using a vibro shifter including a 60 to 300 mesh filter. When the 60 to 300 mesh filter is used, precipitates derived from the deer bones may be effectively removed.

Sixth Operation: Concentration

The filtrate is concentrated at about 35 to about 70° C. and about 0 to about 160 mmHg. Since concentration is performed in this temperature range, heat decomposition of total ganglioside, which is a functional ingredient, may be minimized. In addition, since concentration is performed in such a high vacuum (i.e., about 0 to about 160 mmHg), extraction efficiency of the deer bones may be increased. Moreover, when the concentration process is performed under these conditions, a natural color of the obtained deer bone extract may be retained and generation of bad odors is effectively inhibited.

Seventh Operation: Drying

The concentrate may be dried to form a powder. Here, the drying method is not particularly limited, and a general drying method such as vacuum drying, hot air drying, spray drying, freeze drying, or the like may be used.

One or more embodiments of the present invention will now be described more fully with reference to the following examples and the accompanying drawings. However, these examples are provided for illustrative purposes only and are not intended to limit the scope of the present invention.

### Example 1

### Preparation of Extract Through Normal Pressure Extraction

500 kg of deer bones were added to an extraction vessel, 1,000 L of purified water was added thereto, the deer bones were maintained in the purified water for 4 hours, the resulting solution was discarded, and blood was removed from the deer bones using cold water. These processes were repeated three times. After the blood was removed from the deer bones using cold water, 3,000 L of purified water was added to the deer bones and the deer bones were then subjected to normal pressure extraction at 95 to 100° C. for 4 hours.

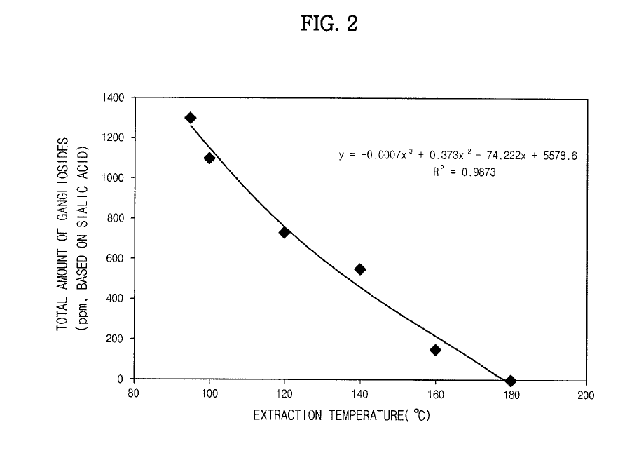

First, to evaluate the relationship between extraction temperature and the total amount of gangliosides, the total amount of gangliosides during extraction at a temperature of 100 to 180° C. for 1 hour was measured and results are shown in FIG. 2. FIG. 2 is a graph showing changes in the total amount of gangliosides during extraction at a temperature from about 100 to about 180° C. for 1 hour. As illustrated in FIG. 2, the deer bone-derived ganglioside has poor thermal resistance. Thus, it is confirmed that high temperature extraction is not desirable if extraction of gangliosides of deer bone is to be maximized.

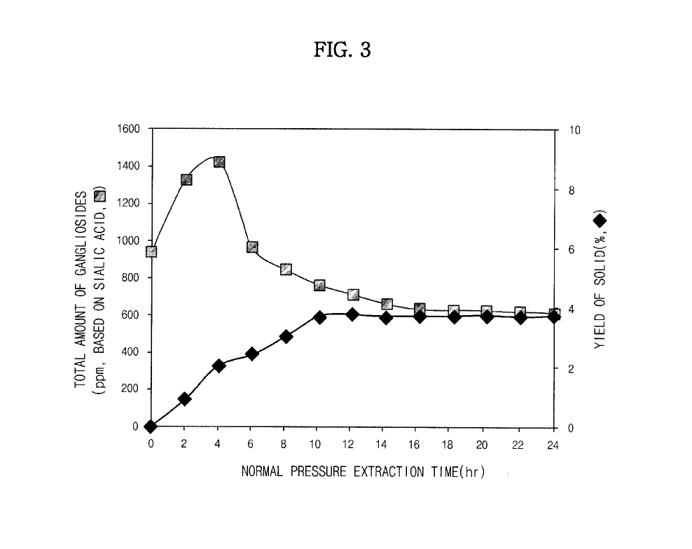

FIG. 3 is a graph showing changes in the total amount and yield of gangliosides when deer bones are subjected to extraction at normal pressure and 100° C. As shown in FIG. 3, as extraction time increases under normal pressure, the total amount of gangliosides and solid yield increase. In addition, the total amount of gangliosides was greatest when the extraction time was 4 hours.

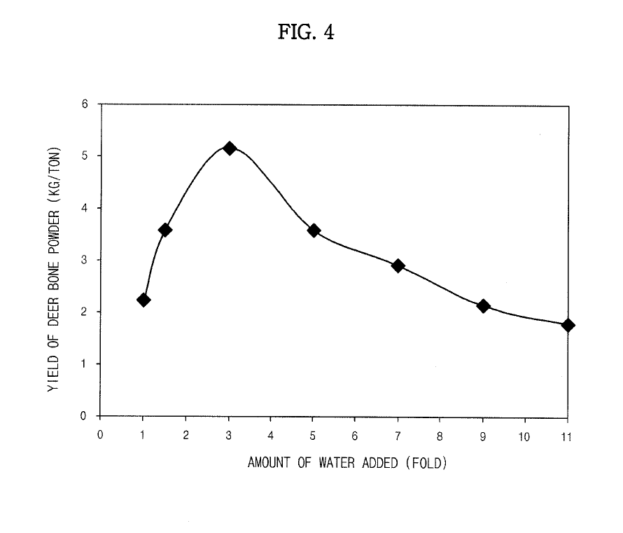

In addition, the relationship between the amount of water added and extraction amount when deer bones were subjected to normal pressure extraction was evaluated and results are shown in FIG. 4. FIG. 4 is a graph showing a yield of deer bone powder per weight (ton) of an extractor when deer bones were subjected to extraction at 100° C. under normal pressure for 4 hours by varying the amount of water added. As shown in FIG. 4, extraction of a mixture of deer bones and purified water in a weight ratio of 1:1.5-7 at normal pressure was effective.

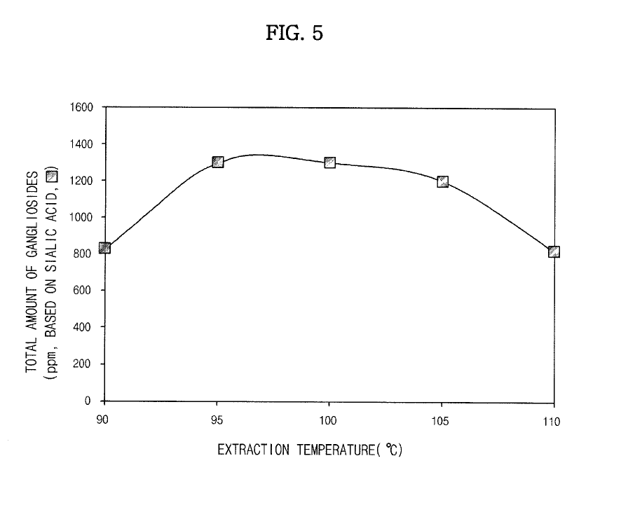

Lastly, the relationship between extraction temperature and the total amount of gangliosides through extraction at normal pressure was evaluated, and results are shown in FIG. 5. FIG. 5 is a graph showing the total amount of gangliosides when deer bones were subjected to extraction at normal pressure for 4 hours by varying an extraction temperature and adding water 6 times the amount of the deer bones. As shown in FIG. 5, extraction at normal pressure and 95 to 105° C. was effective.

### Example 2

### Preparation of Extract Through Extraction Under Pressure

The extract obtained by extraction at normal pressure was transferred to an oil separator, 1,500 L of purified water was added to the remaining deer bones, and the resulting deer bones were subjected to extraction under pressure at 120° C. for 3 hours.

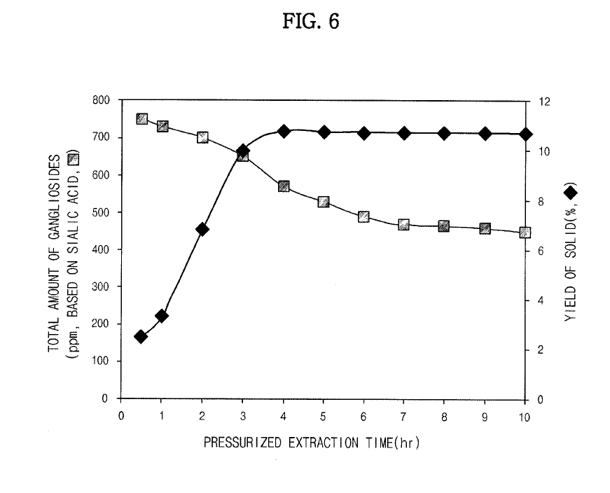

The total amount of gangliosides and solid yield when the deer bones were subjected to extraction under pressure were measured, and results are shown in FIG. 6. As shown in FIG. 6, the solid yield was more significantly improved when the deer bones were subjected to extraction under pressure than when deer bones were subjected to extraction at normal pressure.

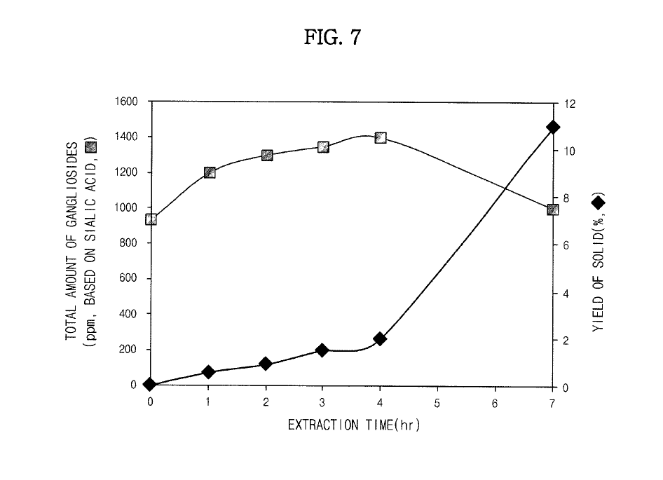

In addition, the total amount of gangliosides and solid yield when deer bones were subjected to extraction at normal pressure and extraction under pressure were measured. By considering conditions for improving both the total amount of gangliosides and solid yield with reference to FIGS. 3 and 6, changes in the total amount of gangliosides and solid yield when the deer bones were subjected to extraction at normal pressure for 6 hours and extraction under pressure for 3 hours were evaluated, and results are shown in FIG. 7. As shown in FIG. 7, it is confirmed that the total amount of gangliosides is greatest in extraction at normal pressure for 4 hours and the solid yield is considerably increased in extraction under pressure for 3 hours. That is, extraction may be performed such that the total amount of gangliosides is first improved by performing extraction on deer bones at normal pressure and solid yield is improved by performing pressurized extraction on the remaining deer bones.

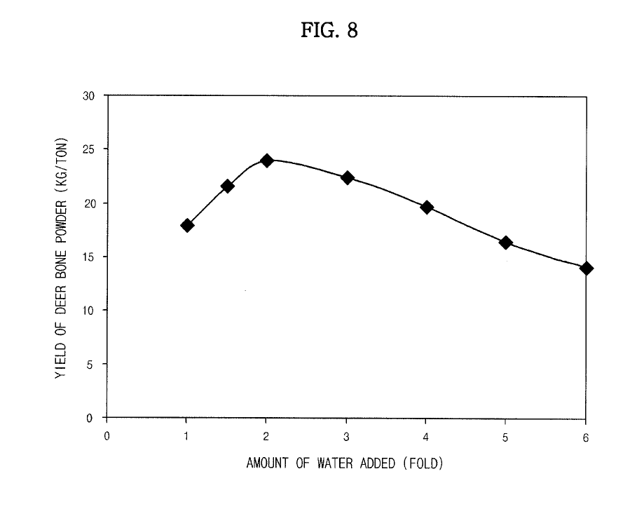

In addition, the relationship between the amount of water added and extraction amount when deer bones were subjected to extraction under pressure was evaluated, and results are shown in FIG. 8. FIG. 8 is a graph showing a yield of deer bone powder per weight (ton) of an extractor when deer bones were subjected to extraction under pressure at 120° C. for 3 hours by varying the amount of water added. As shown in FIG. 8, pressurized extraction of a mixture of deer bones and purified water in a weight ratio of 1:1.5-4 was effective.

### Example 3

### Preparation of Deer Bone Concentrate and Powder

The extracts obtained after normal pressure extraction and pressurized extraction were each maintained in an oil separator at 80° C. for 1 hour and then a lipid layer was removed from each extract. The oil-separated solution was filtered using a vibro shifter including a 200 mesh filter. The filtrate was transferred to a concentrator and then concentrated at 60 mmHg and 55° C. until the solid content reached 40 wt %. Table 1 shows sensory evaluation results of the concentrate obtained by concentrating the filtrate at 35 to 70° C. and 0 to 160 mmHg. As shown in Table 1, the concentrate retained a natural color and generation of bad odors caused by reaction at high temperature was inhibited. A powdering process was performed using a vacuum dryer.

TABLE 1
Sensory evaluation results according to concentration conditions

| | Concentration conditions | |
|---|---|---|
| Sensory | 35~70° C., 0~160 | 70° C., 160 mmHg or |
| Evaluation results | mmHg | higher |
| Natural color* | +++ | + |
| Generation of bad odors | + | +++ |

### Comparative Example 1

A deer bone extract was obtained by extraction at normal pressure alone. The normal pressure extraction was performed under the following conditions. 500 kg of deer bones were added to an extraction vessel, 1,000 L of purified water was added thereto, the deer bones were maintained in the purified water for 4 hours, the resulting solution was discarded, and blood was removed from the deer bones using cold water. These processes were repeated three times. After the blood was removed from the deer bones using cold water, 3,000 L of purified water was added to the deer bones and the deer bones were then subjected to normal pressure extraction at 100° C. for 24 hours. Thereafter, a deer bone concentrate and powder were prepared in the same manner as in Example 3.

### Comparative Example 2

A deer bone extract was obtained by extraction under pressure alone. Pressurized extraction was performed under the following conditions. 500 kg of deer bones were added to an extraction vessel, 1,000 L of purified water was added thereto, the deer bones were maintained in the purified water for 4 hours, the resulting solution was discarded, and blood was removed from the deer bones using cold water. These processes were repeated three times. After the blood was removed from the deer bones using cold water, 1,500 L of purified water was added to the deer bones and the deer bones were then subjected to extraction under pressure at 120° C. for 10 hours. Thereafter, a deer bone concentrate and powder were prepared in the same manner as in Example 3.

### Comparative Example 3

Deer bones were first subjected to extraction under pressure and the remaining deer bones were then subjected to normal pressure extraction to obtain a deer bone extract. Pressurized extraction and normal pressure extraction were performed under the following conditions. 500 kg of deer bones were added to an extraction vessel, 1,000 L of purified water was added thereto, the deer bones were maintained in the purified water for 4 hours, the resulting solution was discarded, and blood was removed from the deer bones using cold water. These processes were repeated three times. After the blood was removed from the deer bones using cold water, 1,500 L of purified water was added to the deer bones and the deer bones were then subjected to extraction under pressure at 120° C. for 3 hours. The resulting extract was transferred to an oil separator, and 3,000 L of purified water was added to the remaining deer bones, which were then subjected to normal pressure extraction at 100° C. for 4 hours. Thereafter, a deer bone concentrate and powder were prepared in the same manner as in Example 3.

### Experimental Example

The total amount of gangliosides and solid yield of the deer bone extracts prepared according to Comparative Examples 1 through 3 were compared with those of the deer bone extract prepared according to Example 3, and comparison results are shown in Table 2.

Referring to Table 2, it is confirmed that the total amount of gangliosides of the deer bone extract of Comparative Example 1 obtained by normal pressure extraction alone is large while the solid yield thereof is considerably low, and the total amount of gangliosides of the deer bone extract of Comparative Example 2 obtained by pressurized extraction alone is low while the solid yield thereof is high. In addition, it is confirmed that the total amount of gangliosides of the deer bone extract of Comparative Example 3 obtained by pressurized extraction, followed by normal pressure extraction is small while the solid yield thereof is high. As for the deer bone extract of Comparative Example 3, gangliosides are thermally decomposed during extraction under pressure, and thus, the total amount of gangliosides is not significantly increased even through normal pressure extraction.

By contrast, when normal pressure extraction is first performed, followed by extraction under pressure as in Example 3, gangliosides may be first extracted by the normal pressure extraction and then pressurized extraction may be performed to increase solid yield.

That is, when normal pressure extraction is performed alone, when pressurized extraction is performed alone, and when pressurized extraction is performed followed by normal pressure extraction, both the total amount of gangliosides and the solid yield are not increased, as compared to when extraction is performed at normal pressure and under pressure as in the embodiment of the present invention.

TABLE 2

| Extraction | | Amount according to extraction time (hr) | | | | | |
|---|---|---|---|---|---|---|---|
| conditions | | 1 | 3 | 5 | 7 | 10 | 24 |
| Comparative | Total amount of | 1120 | | 1180 | 890 | 750 | 600 |
| Example 1 | gangliosides | | | | | | |
| (normal | (ppm) | | | | | | |
| pressure | | | | | | | |
| extraction) | Solid yield (%) | 0.5 | | 2.2 | 2.7 | 3.6 | 3.7 |
| Comparative | Total amount of | 800 | | 500 | 420 | 330 | |
| Example 2 | gangliosides | | | | | | |
| (pressurized | (ppm) | | | | | | |
| extraction) | Solid yield (%) | 4.5 | | 10.9 | 11.1 | 11.2 | |
| Comparative | Total amount of | | 600 | | 670 | | |
| Example 3 | gangliosides | | | | | | |
| (Normal | (ppm) | | | | | | |
| pressure | | | | | | | |
| extraction after | Solid yield (%) | | 9.5 | | 11.2 | | |
| pressurized | | | | | | | |
| extraction) | | | | | | | |
| Example 3 | Total amount of | | | | 1,000 | | |
| | gangliosides | | | | | | |
| | (ppm) | | | | | | |
| | Solid yield (%) | | | | 11 | | |

As is apparent from the above description, a method of preparing a deer bone extract including large amounts of gangliosides and protein includes performing extraction on deer bones at normal pressure and under pressure, filtering the resulting extract, and powdering the filtrate.

By using the method described above, the amount of ganglioside, which is a functional ingredient of deer bones, may be increased, protein of deer bones may be extracted at an optimum level, a deer bone extract may be prepared using an efficient and effective manufacturing process at low manufacturing costs within a short period of time, and a deer bone extract which has high productivity and includes large amounts of factors for growth promotion, joint health, memory improvement, and immunity boost may be obtained.

Although a few embodiments of the present invention have been shown and described, it would be appreciated by those skilled in the art that changes may be made in these embodiments without departing from the principles and spirit of the invention, the scope of which is defined in the claims and their equivalents.

What is claimed is:

1. A method of preparing a deer bone extract, the method comprising:
performing hot water extraction on deer bones at normal pressure;
performing hot water extraction on the deer bones under pressure;
separating oil from the extract;
filtering the oil-separated solution; and
concentrating the filtrate.

2. The method according to claim 1, wherein the hot water extraction at normal pressure is performed at about 95 to about 105° C. for about 3 to about 12 hours after mixing the deer bones and purified water in a weight ratio of about 1:1.5 to about 1:7.

3. The method according to claim 1, wherein the hot water extraction under pressure is performed at about 105 to about 140° C. for about 0.5 to about 12 hours after mixing the deer bones and purified water in a weight ratio of about 1:1.5 to about 1:4.

4. The method according to claim 1, wherein the oil separation is performed using an oil separator after maintaining the extract at about 60 to about 90° C. for about 0.5 to about 4 hours.

5. The method according to claim 1, wherein the filtering is performed using a 60 to 300 mesh filter.

6. The method according to claim 1, wherein the concentrating is performed at about 35 to about 70° C. and about 0 to about 160 mmHg.

7. A deer bone extract prepared using the method according to any one of claims 1 to 6.
